(19) United States
(12) Patent Application Publication
(10) Pub. No.: US 2005/0048120 A1
(43) Pub. Date: Mar. 3, 2005

(54) Dosage form comprising self-destructive membrane

(76) Inventors: David Emil Edgren, Los Altos, CA (US); Robert R. Skluzacek, Newark, CA (US); Shu Li, Newark, CA (US); Patrick S.L. Wong, Burlingame, CA (US); Paul R. Magruder, Mountain View, CA (US)

Correspondence Address: Dewipat Incorporated, 4606 FM 1960 WEST, SUITE 400, HOUSTON, TX, 77069, US

(21) Appl. No.: 10/957,357

(22) Filed: Oct. 1, 2004

Related U.S. Application Data
(62) Division of application No. 09/373,352, filed on Aug. 12, 1999.
(60) Provisional application No. 60/106,939, filed on Nov. 4, 1998.

(51) Int. Cl. A61K 9/26; A61K 9/24; A61K 38/00

(52) U.S. Cl. 424/473; 514/2

(57) ABSTRACT

A sustained release dosage form is disclosed, which dosage form comprises an interior lipophilic membrane, and an exterior hydrophilic membrane comprising a peptide, that surround a composition comprising a drug. The dosage form at the end of its drug delivery period self-ruptures for enhancing its exit from the environment of use.

### REFERENCE TO RELATED APPLICATION

This application claims the benefit of provisional application Ser. No. 60/106,939 filed on Nov. 4, 1998.

### FIELD OF THE INVENTION

The present invention relates to both a novel dosage form and to a novel membrane. More particularly, the invention relates to a sustained release dosage form comprising a drug composition surrounded by an interior membrane possessing lipophilic absorbing properties, and an exterior membrane possessing hydrophilic and self-destructive properties. The invention concerns also a self-destructive membrane comprising a compound possessing a peptide group. The dosage form, after delivering its drug, loses its physical integrity and passes from the environment of use. Further, the invention concerns a method for administering a dose of drug to a patient.

### BACKGROUND OF THE INVENTION

Because of their therapeutic advantage, the majority of drugs are administered orally by a sustained release dosage form. The sustained release dosage form achieves its therapeutic advantage by delivering its drug in transit throughout the gastrointestinal tract.

The orally administered sustained release dosage form begins its gastrointestinal transit first by entering the stomach. The sustained release dosage form remains in the stomach for a length of time, and concomitantly during this transit time it delivers a therapeutically effective dose of drug. Next, after its stomach transit, the sustained release dosage form enters the small intestine. The small intestine is the main site for the absorption of most drugs with its highly effective surface area. Continuing its transit, the sustained release dosage form travels into the large intestine. The large intestine is a rational site for treating localized diseases, and as an entry site for orally administered systemic drugs sensitive to the upper gastrointestinal tract. The gastrointestinal transit time, from mouth-to-anus, among and within subjects, vary significantly. Many dosage forms are retained in the body for at least ten hours, and up to ninety percent are retained for up to twenty-four hours.

The above information indicates, while the prior art dosage forms are useful for the management of health and disease, a serious limitation can be associated with their use. That is, the prior art dosage forms lack the ability to self-destruct. The presence in a sustained release dosage form of the ability to self-destruct would add built-in-safety, as this embodiment would avoid gastrointestinal blockage from the accumulation of intact spent dosage forms, and it would increase the ability of the self-destructed dosage form to leave the body.

Thus, in view of the above presentation, it will be appreciated, since the prior art dosage forms lack the potential ability to alter their physical integrity and avoid possible problems that could be encountered in the gastrointestinal tract, the unexpected availability of a dosage form that overcomes the prior art disadvantage, would represent an advancement, and an improvement, and also have a practical application in the fields of medicine and pharmacy.

### SUMMARY OF THE INVENTION

Accordingly, the present invention is directed to a novel and unique dosage form comprises means for self-destruction for enhancing its passing from the environment of use.

Another aspect of the present invention is to provide a sustained release dosage form comprising an exterior membrane comprising a compound possessing a peptide group for providing self-destruction in an environment of use.

Another purpose of the invention is to provide a sustained release dosage form comprising an interior membrane and an exterior membrane that cooperate for facilitating the passage of the dosage form from an environment of use.

Another purpose of the invention is to provide a sustained release dosage form that can transit the gastrointestinal tract free of blockage or retention thereof.

Another purpose of this invention is to provide a membrane comprising means for self-destruction in a fluid environment of use.

Another purpose of the invention is to provide a dosage form characterized by ease of manufacture, which dosage form can administer a sustained release of drug up to 30 hours and thereby provide a plasma drug concentration proportional to the dose of delivered drug.

Other advantages of this invention will be more apparent to those of ordinary skill in the drug delivery art taken in conjunction with the drawings, the specification, and the claims.

### BRIEF DESCRIPTION OF DRAWINGS

In the accompanying drawing figures, which are not drawn to scale, but are set forth to illustrate embodiments of the invention, the drawing figures are as follows:

Drawing FIG. 1 is a general view of a sustained release dosage form provided by the invention.

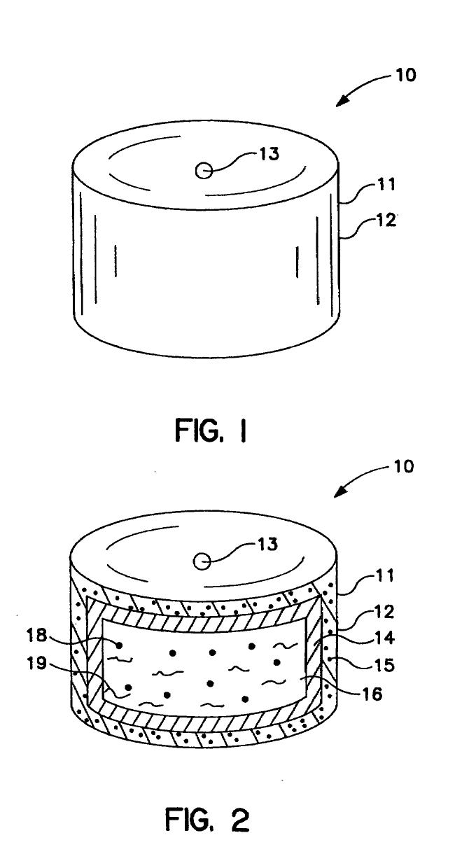

Drawing FIG. 2 is an opened view of drawing FIG. 1 illustrating the dosage form comprising an exterior membrane, an interior membrane, and an interior compartment comprising a therapeutically active drug composition.

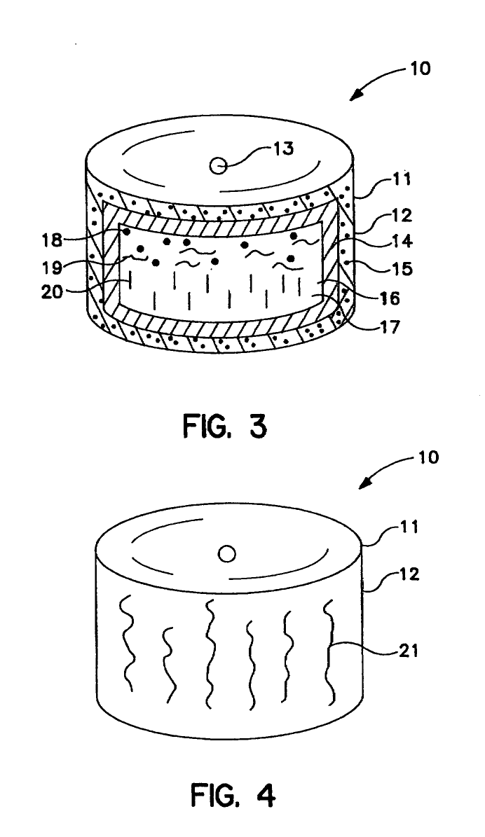

Drawing FIG. 3 is an opened view of drawing FIG. 1 depicting the interior compartment comprising a therapeutically active drug composition and a composition comprising means for changing from a rested state to an expanded state.

Drawing FIG. 4 is a closed view of the dosage form of FIG. 1, at/or near the end of its drug delivery period depicting developed structural imperfections in the exterior wall of the dosage form for enhancing its exit from the environment of use.

In the drawing figures and in the specification, like parts in related figures are identified by the numbers. The terms appearing earlier in the specification are described later in the specification.

### DETAILED DESCRIPTION OF DRAWINGS

The phrase a “sustained release” as used for the purpose of this invention denotes a dosage form that releases a drug during several hours, typically more than 3 hours up to 30 hours. The term sustained release also means for the purpose of the present invention a therapeutically effective amount or dose of drug is released from the dosage form at a controlled rate such that therapeutically effective blood levels of the drug are maintained up to 30 hours. The phrase “therapeutically effective amount or dose” as used therein indicate the amount or dose delivered to the patient produces a therapeutic effect, or a pharmacologic benefit in a patient. The term “hydrophilic” as used herein denotes a material such as a composition or a composition comprising a polymer having a strong affinity for and readily associates with when contacted by an aqueous medium such as water and biological fluids. The term “lipophilic” as used herein denotes a material having an affinity to sorb or take-up lipoids or fats from an environment. The term “self-destruct” and “disintegrate” as used herein denotes the ability to break, to fracture, to rupture, to fissure, and thereby change its physical structure from the original unit manufacture into elements, or parts or into a lesser size. The phrase “controlled release” as used herein denotes the delivery of drug at a prolonged period of time. The term “semipermeable” denotes a composition, a material, or a polymer that is permeable to the passage of drug. The phrase “compound possessing at least one peptide moiety” as used herein denotes a biopolymer that describes a polymer produced of biological or vegetable origin. The biopolymer comprises a compound possessing a peptide bond, including polypeptides and proteins joined by a peptide moiety.

Turning now to the drawing figures in detail, which are examples of sustained release dosage provided by this invention, one sustained release dosage form of the invention is seen in drawing FIG. 1. In FIG. 1, dosage form 10 comprises a body 11, comprising an exterior membrane 12 and an exit 13. Exit 13 connects the exterior of dosage form 10 with the interior of dosage form 10.

In drawing FIG. 2, dosage form 10 is seen in opened section for illustrating the structure and the compositions of dosage form 10. Dosage form 10 comprises a body 11, an exterior membrane 12, or second membrane 12, an exit passageway 13, an interior membrane 14, or a first membrane 14, and an internal compartment 16 or space formed and defined by exterior membrane 12 and interior membrane surrounding compartment 16. The phrase first membrane denotes the first membrane coated around a drug formulation; and the second membrane denotes the second membrane coated around and in contact with the first membrane. Exterior membrane comprises 35 wt % (weight percent) to 70 wt % of a semipermeable polymer permeable to the passage of fluid and impermeable to the passage of a therapeutically active agent, 10 wt % to 40 wt % of a plasticizer, 0 wt % to 10 wt % of a surfactant, and in a present manufacture 0.01 wt % to 10 wt % of the surfactant, and 20 wt % to 35 wt % of a compound 15 possessing at least one peptide moiety, with the weight of all membrane 12 materials equal to 100 wt %. The interior membrane comprises 35 wt % to 70 wt % of a lipophilic attracting material, 25 wt % to 65 wt % of a flux enhancer, and 0 wt % to 10 wt % of a surfactant, in a present manufacture 0.01 wt % to 10 wt % of the surfactant, with the weight of all interior membrane ingredients equal to 100 wt %. Interior compartment 16 comprises a drug 18 and a pharmaceutically acceptable carrier 19 for transporting drug 18 through exit 13 from dosage form 10. Compartment can contain additional compositional forming ingredients such as a surfactant for wetting the drug, a nontoxic colorant for identifying the drug and for making the delivery of a drug visible.

Drawing FIG. 3 depicts dosage form 10 in opened section. In FIG. 3, dosage form 10 comprises body 11, exterior membrane 12 comprising compound 15, exit passageway 13, interior membrane 14 and interior compartment 16. Interior compartment 16 comprises a therapeutic composition comprising drug 18 and a pharmaceutically acceptable carrier 19. Interior compartment also comprises composition 17, which is an expandable, displacement layer 17, comprising a fluid imbibing and expandable hydrophilic hydrogel 20. Expandable layer 17 is in contact with the therapeutic composition, and during operation layer 17 imbibes, absorbs fluid and expands, for displacing the therapeutic composition comprising drug 18 from dosage form 10.

Drawing FIG. 4 illustrates sustained release dosage form 10 at/or near the end of its drug delivery period. Dosage form 10, when in a fluid environment of use, such as the gastrointestinal tract of an animal, allows aqueous and biological fluid to enter into exterior membrane 12, and to leach or fluid extract a compound 15 from membrane 12 to impart self-destruction properties to membrane 12. The fluid flux of fluid into hydrophilic membrane 12, and the fluid extraction of compound 15, cause membrane 12 to be mechanically weakened by the developments of cracks, fissures and/or rupture that permit membrane 12 to break-open and collapse. This mechanical activity, and its accompanying loss of integrity of membrane 12, exposes interior membrane 14 to biological, endogenous lipids present in the gastrointestinal tract. Interior membrane 14, since it is porous and lipophilic attracting, sorbs the endogenous lipids which soften membrane 14, thereby making membrane 14 flaccid, weak and lowering its glass transition temperature. Additionally, the churning action of the gastrointestinal tract, joined with the expanding and swelling action of the hydrophilic displacement composition 20 dosage form 10, cause membrane 14 to split, burst, collapse, or otherwise lose its mechanical integrity. The combined physical actions of membranes 12 and 14, joined with the expansion-pressure of the displacement layer provide a residual dosage form 10 that is excreted smaller than its original size.

### DETAILED DESCRIPTION OF INVENTION

Dosage form 10, as seen in FIGS. 1 to 3, are useful for establishing therapeutic drug levels in the blood, including the plasma, proportional to the administered dose, for therapy. Dosage form 10 can embrace a multiplicity of shapes, including the shape of a standard conventional tablet, the sustained release dosage form can embrace the shape of a caplet, or a buccal, or a sublingual dosage form. The sustained release dosage form of the invention provides sustained-continuous drug delivery up to 30 hours, greater than conventional noncontrolled immediate release tablets, or noncontrolled-nonsustained release tablets, and/or noncontrolled-nonsustained release capsules that exhibit dose-dumping of their drug. Dose-dumping dosage forms release drug content in less than one hour.

Sustained release dosage form 10 of the invention comprises exterior membrane 12. Membrane 12 comprises a composition that is non-toxic, membrane 12 does not adversely affect an animal, including a human, or the components of dosage form 10. Compositions for forming membrane 12, are in one embodiment, comprised of a member selected from the group consisting of a cellulose ester polymer, a cellulose ether polymer, and a cellulose ester-ether polymer. The cellulose polymers have a degree of substitution DS, on the anhydroglucose unit, from greater than 0 up to 3 inclusive. By “degree of substitution” is meant the average number of hydroxyl groups originally present on the anhydroglucose unit comprising the cellulose polymer that are replaced by a substituting group. Representative membrane 12 polymers comprise a member selected from the group consisting of cellulose acylate, cellulose diacylate, cellulose triacylate, cellulose acetate, cellulose diacetate, cellulose triacetate, mono-, di-, and tricellulose alkylates, mono-, di, and tricellulose alkenylates, and mono-di-, and tricellulose aroylates. Exemplary polymers include cellulose acetate having a DS of up to 1 and an acetyl content of up to 31%; cellulose acetate having a DS of 1 to 2 and any acetyl content of 21 to 35%; cellulose acetate having a DS of 2 to 3 and an acetyl content of 35 to 44.8%; and the like. More specific cellulosic polymers comprise cellulose propionate having a DS of 1.8, a propyl content of 39.2 to 45% and a hydroxyl content of 2.8 to 5.4%; cellulose acetate butyrate having a DS of 1.8, an acetyl content of 13 to 15% and a butyryl content of 34 to 39%; cellulose acetate butyrate having a acetyl content of 2 to 29%, a butyryl content of 17% to 53% and a hydroxyl content of 0.5 to 4.7%; cellulose triacylates having a DS of 2.9 to 3, such as cellulose trivalerate, cellulose trilaurate, cellulose tripalmitate, cellulose trisuccinate, and cellulose trioctanoate; celluloses diacylate having a DS of 2.2 to 2.6, such as celluose disuccinate, cellulose dipalminate, cellulose dioctanoate, cellulose dipentanoate, co-esters of cellulose, such as cellulose acetate butyrate, and cellulose acetate propionate.

Additional semipermeable polymers comprise acetaldehyde dimethylcellulose acetate; cellulose acetate ethylcarbamate; cellulose acetate methylcarbamate; cellulose diacetate propylcarbamate; cellulose acetate diethylaminoacetate; semipermeable polyamide; semipermeable poluyurethane; semipermeable sulfonated polystyrene; semipermeable crosslinked selective polymer formed by the coprecipitation of a polyanion and polycation, as disclosed in U.S. Pat. Nos. 3,173,876; 3,276,586; 3,541,005; 3,541,006 and 3,546,876; semipermeable polymers as disclosed by Loeb and Sourirajan in U.S. Pat. No. 3,133,132; semipermeable, lightly crosslinked polystyrenes; semipermeable crosslinked poly(sodium styrene sulfonate); semipermeable cross-linked poly(vinylbenzyltrimethyl ammonium chloride); and semipermeable polymers possessing a fluid permeability in the range of 2.5×10⁻⁸ to 5×10⁻² (cm²/hr·atm), expressed per atmosphere of hydrostatic or osmotic pressure differences across the semipermeable wall. The polymers are known to the polymer art in U.S. Pat. Nos. 3,845,770; 3,916,899 and 4,160,020; and in Handbook of Common Polymers, by Scott, J. R. and Roff, W. J., 1971, CRC Press, Cleveland, Ohio.

Membrane 12 comprises a plasticizer that can increase the flux into membrane 12 by increasing the aqueous diffusion coefficient of membrane 12. Representative of plasticizer for membrane 12 comprise a member selected from the group consisting of glycerin, triacetin also known as glycerol triacetate, adipic acid plasticizer, azelaic acid plasticizer, citric acid plasticizer, triethyl citrate also known as citric acid ethyl ester, acetyl triethyl citrate, tributyl citrate, acetyl tributyl citrate, butyryl trihexyl citrate, glycol plasticizers including polyethylene glycol, diethylene glycol dipelargonate, triethylene glycol di(2-ethylbutrate), di-n-butyl sebacate, phthalate plasticizers represented by disobutyl phthalate, undecyldoceyl phthalate and disobutyl phthalate, tricresyl phosphate, cellulose nitrate, dimethlamide, methyl ricinoleate, acetyl triethyl hexyl citrate, methyl phthalyl ethyl glycolate, ethylene glycol diproprionate, monoacetin, diacetin, tributyrin, polyester of diethylene glycols and succinic acid, sorbitol, and diphenylactyl phosphate. Plasticizers are known in Encyclopedia of Chemical Technology, 4th Ed., Vol. 19, pg. 258 to 260, (1996), published by Wiley-Interscience Publication.

Membrane 12 comprises optionally a surfactant that functions to increase the aqueous flux into membrane 12. The surfactant serves to reduce interfacial tension between constituents, and the surfactant aids in membrane 12 decreasing its density and mechanical integrity. The surfactants useful for formulating membrane 12 for the purposes of this invention comprise anionic, amphoteric, cationic and nonionic surfactants. Membrane 12 of this invention in a present manufacture comprises a nonionic surfactant. Representative of surfactants include polyoxyethylene sorbitan fatty acids commercially available as Polysorbate® 20 to 120 exemplified by polyoxyethylene 20 sorbitan monolaurate, polyoxyethylene 20 sorbitan monopalmitate, polyoxyethylene 20 sorbitan monostearate, polyoxyethylene 20 sorbitan tristearate, polyoxyethylene 20 sorbitan monooleate, polyoxyethylene 20 sorbitan monoisostearate, and polyoxyethylenated stearic acid comprising 8 moles of ethylene oxide available as Myrj® 45. The surfactants are available from ICI Americas, Inc., Wilmington, Del.

Exterior membrane 12 comprises a compound possessing at least one peptide bond or peptide linking group. The term peptide comprises natural polymers identified further as a protein polymer. The protein polymer comprises monomers joined by a peptide link to provide molecular weights of 1500 to 350,000. The proteins can be of vegetable or of animal origin. Proteins comprising amino acids joined by a peptide bond for the purpose of this invention are represented by reticulin, silk, keratin, casein, lactoglobulin, prolamine, gluten, albumin, elastin, soy protein, globulin, gelatin, collagen, and zein. The proteins can be compound with other membrane 12 forming ingredients in submicron and in micron size. The protein can be micronized using standard micronizer such as the fluid-energy mill, microfluidizer the fluidized-bed jet, the micronizer to produce a submicron to a micron size product with an average size of 0.01 to 50 microns. Procedures for micronizing a polymer are disclosed in Perry's Chemical Engineers' Handbook, pages 20-22 to 20-48, (1997), 7th Ed. The further preparation of micron sized particles is presented in U.S. Pat. No. 4,177,256.

Interior membrane 14 comprises two or more membrane 14 forming ingredients that act together to provide a lipophilic membrane that function as a single membrane. The interior membrane 14 comprises an alkyl cellulose, a long-chain polymer consisting of anhydroglucose units joined together by acetal linkages. Representative of alkyl cellulose is ethyl cellulose. Ethyl cellulose comprises anhydroglucose units having three replaceable hydroxyl groups that are substituted by 1.25 to 3.00 ethoxyl groups per unit to provide an ethoxyl content of 44 to 51%, and a viscosity of 3 to 350 centipoise. Ethyl cellulose is a nontoxic polymer, a poly(ethyl cellulose) comprising a 3,000 to 450,000 molecular weight, it is insoluble in water, and insoluble in the fluids of the gastrointestinal tract.

Membrane 14 comprises additionally a flux enhancer selected from the group consisting of hydroxyalkylcellulose wherein alkyl is 1 to 6 carbon atoms represented by methyl, ethyl, propyl, butyl, pentyl, and hexyl. A representative of a non-toxic flux enhancer for providing inside liposorbing membrane 14 is nonionic water soluble polymer hydroxyethylcellulose. Hydroxyethylcellulose possesses a 30,000 to 1,500,000 viscosity-average molecular weight and a viscosity of 50 to 7,000 cps (centipoise); and a hydroxypropylcellulose nonionic water-soluble cellulose ether comprising a 10,000 to 2,750,000 viscosity average molecular weight possessing a viscosity of 75 to 6,500 cps. The hydroxyalkylcellulose ethers are commercially available from Aqualon Co., Wilmington, Del.

Interior membrane 14 optionally comprises a surfactant represented by polyoxyethylene stearate identified for example as Polyoxyl® 8 stearate wherein the number 8 refers to the polymer length in oxyethylene units. Representative of other surfactants include Polyoxyl 4 stearate, Polyoxyl 8 stearate known also as Myrj 45; Polyoxyl 20 stearate or Myrj® 49; Polyoxyl 30 stearate or Myrj® 51; Polyoxyl 40 stearate or Myrj® 52; Poloxyl 50 stearate or Myrj® 53; Poloxyl 100 stearate Myrj® 59; Poloxyl 4 distearate; Poloxyl 150 distearate; nonionic polyoxyethylene alkyl ethers such as Polyoxyl 2 cetyl ether, Poloxyl 23 lauryl ether; polyoxyethylene castor oil surfactants such as Polyoxyl 35 castor oil or Cremophor® EL; Polyoxyl 40 hydrogenated castor oil or Cremophor® RH40; and polyoxyethylene sorbitan fatty acid esters. The surfactants are available from BASF Inc., Mt. Olive, N.J.; and ICI Americas, Inc., Wilmington, Del.

The term drug 18, as used for the purpose of this invention includes medicine, nutrient, vitamin, food supplement, and other beneficial agents that provide a therapeutic effect or a benefit to animals, including a warm-blooded animal, human, farm animal, and zoo animal. Representative drugs include acetaminophen, acyclovir, albuterol sulfate, alprazolam, amitriptylen hydrochloride, amoxicillin triphydrate, amphetamine sulfate, aspirin, bacitracin zinc, beclomethasone dipropionate, betamethasone sodium, captopril, diltiazem, caphalexin, cimetidine, clonidine hydrochloride, dextromethorphan hydrobromide, enalapril, enalaprilat, ethinyl estradiol, guaifensin, enitabas, hydrochlorothiazide, imipramine hydrochloride, ketoprofen, levodopa, lorazepan, melatonin, methscopolamine, naproxen, nitroglycerin, phenylephrine hydrochloride, ranitidine, scopolamine, selegiline, tacrine, valproic acid, infliximab for treating Crohn's disease, nelfinavir for anti-HIV therapy, nevirapine for anti-HIV therapy, sildenafil for the treatment of erectile dysfunction, toremifene for therapy for breast cancer in postmenopausal women, repaglinide for treatment of type-2 diabetes mellitus, tiagabine anticonvulsant, trovalfoxacin antibacterial, alatrofloxacin antibacterial, lobaplatin antineoplastic, orlistat treatment of obesity, raloxifene prevention of vertebral fractures in postmenopausal women, fluiticasone propionate for inflammatory bowel disease, verapamil, zidovudine, vancomycin, valoxifene, lisinopril, clomipramine, pergolide, mesalazine, omeprazole, oxybutynin, saquinavir, ritonavic, indinavir, and nelfinavir. The dose of drug 18 includes the pharmaceutically acceptable salt of the drug. The dose of drug 18 in sustained release dosage form 10 is 100 ng to 750 mg.

Dosage form 10, in interior compartment 16, comprises a pharmaceutically acceptable carrier. Representative of the pharmaceutically acceptable carrier is a polymer hydrogel comprising a maltodextrin polymer comprising the formula (C₆H₁₂O₅)ₓ.H₂) wherein X is 3 to 7,500 and the maltrodextrin comprises a 500 to 1,250,000 number average molecular weight; a poly(alkylene oxide) represented by a poly(ethylene oxide) and a poly(propylene oxide) having a 50,000 to 750,000 weight average molecular weight, and more specifically represented by a poly(ethylene oxide) of at least one of 100,000, 200, 000, 300,000 or 400,000 weight-average molecular weight; an alkali carboxyalkylcellulose wherein the alkali is a member selected from the group consisting of sodium, potassium, calcium, or lithium, the alkyl is a member selected from the group consisting of methyl, ethyl, propyl, butyl, or pentyl, of 10,000 to 1,250,000 weight average molecular weight; a poly(acrylic acid) including poly(acrylates) and poly(methacrylates), acrylic copolymers, and poly(acrylamide) possessing a 10,000 to 1,750,000 weight average molecular weight; and a copolymer of ethylene-acrylic acid, including methacrylic and ethacrylic acid of 10,000 to 500,000 number average molecular weight. The drug formulation comprises 5 mg to 425 mg of the pharmaceutically acceptable carrier for aiding in delivering a drug from dosage form 10. The polymer hydrogel carrier exhibits an osmotic pressure gradient across the bimembrane comprising the exterior membrane and the interior membrane, thereby imbibing fluid into dosage form 10 to form a solution or a suspension containing drug that is hydrodynamically and osmotically delivered from sustained release dosage form 10.

Sustained release dosage form 10, in drawing FIG. 2, comprises 0.0 mg to 450 mg of an osmotically effective solute, in a present embodiment 1 mg to 450 mg also known as osmotically effective compound, osmotic compound, or osmagent. The osmagent exhibit a concentration gradient across the exterior and the interior membrane, thereby imbibing fluid into dosage form 10. The imbibed fluid creates hydrodynamic and osmodynamic energies that aid in delivering drug 18 formulation for dosage form 10.

Representative of osmagents include magnesium sulfate, magnesium chloride, sodium chloride, lithium chloride, potassium sulfate, sodium sulfate, lithium sulfate, potassium chloride, potassium acid phosphate, mannitol, sorbitol, urea, inositol, succinic acid, tartaric acid, raffinose, sucrose, glucose, and fructose. The osmagent can be in any form such as particle, crystal, pellet, strip, film, or granule.

Dosage form 10 can comprise additional drug formulation ingredients, such as a binder. The binder imparts cohesive qualities to the drug formulation. Representative of binder materials for this invention useful as binders comprise a member selected from the group consisting of starch, gelatin, molasses, a vinyl polymer comprises a 5,000 to 350,000 viscosity-average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate, and other binders such as methylcellulose, hydroxypropylcellulose, hydroxypropylmethylcellulose, and mixtures of binders. The binders can be used as a solution, or in a dry form to prepare the drug formulation. The drug formulation comprises 0 to 100 mg of a binder, and in one manufacture from 0.01 to 25 mg of the binder.

Dosage form 10 comprises a lubricant used during manufacture of the drug formulation to prevent sticking to die walls or punch faces, generally to lessen adhesion. The lubricants are selected from the group consisting of sodium stearate, oleic acid, potassium oleate, caprylic acid, sodium stearyl fumarate, magnesium palmitate, calcium palmitate, sodium suberate, potassium laureate, stearic acids, salts of fatty acids, salts of alicyclic acids, salts of aromatic acids, oleic acid, palmitic acid, a mixture of salts of fatty acid, a mixture of salts of alicylic acid, or a mixture of magnesium stearate and stearic acid, glyceryl monostearate, glyceryl palmitostearate, hydrogenated castor oil, and talc. The concentration of lubricant in the drug 18 formulation is generally 0 to 3 weight percent, or in another embodiment 0.1 mg to 20 mg.

Drawing FIG. 3 depicts dosage form 10 in opened section, illustrating space 16 comprising a drug formulation and an expandable composition. The drug formulation, is as described in detail in drawing FIG. 2, and that detailed description is incorporated herein by reference. The expandable composition in drawing FIG. 3 comprises 10 mg to 450 mg of an expandable osmopolymer. The omopolymer in the expandable composition possesses a higher molecular weight than the hydrogel polymer in the drug formulation. The osmopolymer comprises a member selected from the group consisting of a polyalkylene oxide and a carboxyalkylcellulose. The polyalkylene oxide possesses a 1,000,000 to 10,000,000 weight-average molecular weight. Representative of polyalkylene oxide include a member selected from the group consisting of polymethylene oxide, polyethylene oxide, polypropylene oxide, polyethylene oxide having a 1,000,000 molecular weight, polyethylene oxide comprising a 2,000,000 molecular weight, polyethylene oxide comprising a 3,000,000 to 5,000,000 molecular weight, polyethylene oxide comprising a 7,000,00 to 7,800,000 molecular weight, cross-linked polymethylene oxide possessing a 1,000,000 molecular weight, and polypropylene oxide possessing a 1,000,000 molecular weight, and polypropylene oxide of 1,200,000 molecular weight. Typical osmopolymer carboxyalkylcellulose in the expandable composition comprises a 200,000 to 7,250,000 weight-average molecular weight. Representative carboxyalkycellulose comprises a member selected from the group consisting of alkali carboxyalkylcellulose, sodium carboxymethylcellulose, calcium carboxymethylcellulose, potassium carboxymethylcellulose, sodium carboxyethylcellulose, lithium carboxyalkylhydroxyalkylcellulose, sodium carboxyethycellulose, potassium carboxyalkyhydroxyalkylcellulose, carboxymethylhydroxyethylcellulose, carboxyethylhydroxyethylcellulose and carboxymethylhydroxypropylcellulose. The osmopolymers used for the push-expandable push composition exhibit an osmotic pressure gradient across the bicoated membrane wall. The osmopolymers imbibe fluid into dosage form 10, thereby swelling, or expanding as a hydrogel or osmogel whereby, they push the drug from the osmotic sustained release dosage form.

The expandable composition comprises 0 mg to 450 mg, and in one embodiment 0.5 mg to 450 mg of an osmotically effective compound. The osmotically effective compounds are known also as osmagents and as osmotically effective solutes. They imbibe an environmental fluid, for example, fluid from the gastrointestinal tract, into dosage form 10 for contributing to the delivery kinetics of the expandable composition. Representative of osmotically active compounds comprise a member selected from the group consisting of osmotic salts, such as sodium chloride, potassium chloride, magnesium sulfate, lithium phosphate, lithium chloride, sodium phosphate, potassium sulfate, sodium sulfate, potassium phosphate, osmotic carbohydrates such as mannitol, sorbitol, fructose and maltose, and other osmagents such as urea, succinic acid, tartaric acid, potassium acid phosphate, citric acid, a mixture of sodium chloride and glucose, and a mixture of sodium chloride and urea.

The expandable composition comprises 0 mg to 75 mg of a suspending agent. The suspending agent adds additionally osmotic imbibing and osmotic driving energy to sustained release dosage form 10. The dual acting suspending-osmotic energy agents are represented by hydroxypropylalkylcellulose comprising an alkyl of 1 to 7 carbons, straight or branched, with the hydroxypropylalkylcellulose possessing a 9,000 to 450,000 number-average molecular weight. The hydroxypropylalkylcellulose is represented by a member selected from the group consisting of hydroxypropylmethylcellulose, hydroxypropylethylcellulose, hydroxypropylbutylcellulose and hydroxypropylpentylcellulose. The expandable composition optionally comprises a hydroxyalkylcellulose. The hydroxyalkylcellulose is a viscosity-increasing agent and it comprises a member selected from the group consisting of hydroxymethylcellulose, hydroxyethylcellulose, hydroxypropylcellulose and hydroxybutylcellulose comprising a 7,500 to 150,000 viscosity-average molecular weight. The viscosity-increasing agent imparts cohesiveness to the expandable composition for making it easier and more effective for operating in the sustained release dosage form. The concentration of hydroxyalkylcellulose in the expandable composition is 0.0 mg to 60 mg.

A lubricant is formulated into the expandable composition with typical lubricant and the concentration are as set forth previously.

An antioxidant is optionally present in the expandable composition to inhibit oxidation of ingredients in the expandable composition. The expandable composition and the drug formulation are in dosage form 10 in FIG. 3 in contacting layer arrangement. The expandable composition comprises 0.0 to 5 mg of an antioxidant. Representative antioxidants comprise a member selected from the group conisting of ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiary butylphenol, alpha-tocopherol, and propylgallate.

The dosage form 10, in the drug formulation, or in the expandable composition, or in both the drug formulation and the expandable composition can comprise 0 mg to 5 mg or the same, or a different nontoxic colorant. The colorant makes dosage form 10 more esthetic in appearance, and it serves to identify the dosage form during manufacture and during therapy. The nontoxic colorants include Food and Drug Administrations Colorant (FD&C), such as FD&C No. 1 blue dye, FD&C No. 4 red dye, FD&C yellow No. 5, FD&C yellow dye No. 6, FD&C blue No. 2, FD&C green No. 3, FD&C cranberry red No. 40, red ferric oxide, yellow ferric oxide, black ferric oxide, titanium dioxide, carbon black, indigo, and Opadry® comprising polymers, polysaccharides, cellulose, starch, and dye commercially available from Coloron, West Point, Pa.

Dosage form 10, comprises another drug delivery manufacture provided by the invention. Dosage form 10 in this manufacture comprises an overcoat, not shown, on the outer surface of the wall of dosage form 10. The overcoat is a therapeutic composition comprising 0 to 75 mg of drug and 0.5 to 275 mg of a pharmaceutically acceptable carrier selected from the group consisting of an alkylcellulose, hydroxyalkylcellulose and hydroxypropylalkylcellulose. The overcoat more specifically is represented by methylcellulose, ethyl cellulose, hydroxyethylcellulose, hydroxybutylcellulose, hydroxypropylcellulose, hydroxypropylmethylcellulose, hydroxypropylethylcellulose and hydroxypropylbutylcellulose. The overcoat can be formulated also with 0 wt % to 50 wt % of a member selected from the group consisting of plasticizer, opacificer, colorant, or antitack agent. The overcoat is formulated with 0-50 weight percent of a plasticizer, opacificer, colorant, and antitack agents. The overcoat provides therapy immediately as the overcoat dissolves or undergoes dissolution in the presence of gastrointestinal fluid and concurrently therewith delivers the drug into the gastrointestinal tract for immediate drug therapy in up to thirty minutes.

Dosage form 10, manufactured as an osmotically controlled-release, zero-order dosage form, comprises at least one passageway 13. The phrase “controlled-release” as used therein indicates that control is exercised over both the duration and profile of the drug release pattern. The expression “zero order” as used herein denotes the delivery of drug by the dosage form is constant and independent of the drug and independent of a receiving biological environment of use. The expression “passageway” as used for the purpose of this invention, includes aperture, hole, orifice, bore, pore, porous element through which a drug can be hydrodynamically and osmotically pumped, diffuse or migrate through a fiber, capillary tube, porous overlay, porous insert, microporous member, hydrogel composition, or porous composition. The passageway 13 includes also a compound that erodes or is leached from the membrane 12 in the fluid environment of use to produce at least one passageway. Representative compounds for forming a passageway include erodible poly(glycolic) acid, or poly(lactic) acid in the membrane; a gelatinous filament; a water-removable poly(vinyl alcohol); leachable compounds such as fluid-removable pore-forming polysaccharides, peptides, proteins, acids, salts or oxides. A passageway can be formed by leaching a compound such as a porosigen from membrane 12, represented by sorbitol, sucrose, lactose, maltose or fructose, to form a controlled-release dimensional pore-passageway. The passageway can have any shape, such as round, triangular, square and elliptical, for assisting in the controlled-metered release of drug 18 from the dosage form. The dosage form can be manufactured with one or more passageways in spaced-apart relation on one or more surfaces of the dosage form. A passageway and equipment for forming a passageway are disclosed in U.S. Pat. Nos. 3,845,770 and 3,916,899 by Theeuwes and Higuichi; in U.S. Pat. No. 4,063,064 by Saunders et al.; in U.S. Pat. No. 4,088,864 by Theeuwes et al.; and in U.S. Pat. No. 5,718,700 by Edgren, Skluzacek, Barclay and Bhatti. Passageways comprising controlled-release dimensions sized, shaped and adapted as a releasing-pore formed by aqueous leaching to provide a releasing-pore of a controlled-release rate are disclosed in U.S. Pat. Nos. 4,200,098 and 4,285,987 by Ayer and Theeuwes.

### Description for Manufacturing the Composition and Dosage Form of the Invention

The first membrane and the second membrane of the dosage form can be formed by using the air coating procedure. This procedure consists in suspending and tumbling the membrane forming composition in a current of air and membrane-forming composition until a membrane, the interior membrane, is applied to the drug compartment. The air suspension procedure is well suited for independently forming a membrane. The air suspension procedure is described in U.S. Pat. No. 2,799,241; J. Am. Pharm. Assoc., Vol. 48, pp. 451-454 (1959); and id., Vol. 49, pp. 82-84 (1960). The membrane can be formed with a membrane forming composition in a Wurster® air suspension coater using an organic solvent, such as acetone-water cosolvent 90:10 (wt;wt) with 1-0 wt % to 7 wt % polymer solids. The hydrophilic membrane and the lipophilic membrane both are manufactured by this process. An Aeromatic® suspension coater can be used for applying the hydrophilic membrane and the lipophilic membrane. An air suspension coater can be used for applying successive membranes.

Other membrane forming techniques, such as pan coating, can be used for providing the dosage form. In the pan coating system, membrane-forming compositions are deposited by successive spraying of the composition or for the bilayered membrane-arrangement, accompanied by tumbling in a rotating pan. A larger volume of cosolvent can be used to reduce the concentration of polymer solids to produce a thinner coat. Finally, the membrane or the membranes of the membrane coated compartments are laser or mechanically drilled, and then dried in a forced air or humidity oven for 1 to 3 days or longer to free, dosage form of the solvent. Generally, the membranes formed by these techniques have a thickness of 1 to 20 mils (0.0254 to 0.508 μm) with a preferred thickness of 1 to 6 mils (0.0254 to 0.152 nm).

The dosage form of the invention in another embodiment is manufactured by standard manufacturing techniques. For example, in one manufacture the beneficial drug and other ingredients comprising a therapeutic composition or comprising the first composition layer facing the exit means are blended, or the ingredients are blended then pressed, into a solid layer. The drug and other ingredients can be blended dry or with a solvent and formed into a solid or semisolid formed by conventional methods such as ball-milling, calendaring, stirring, or roll-milling and then pressed into a selected shape. The drug layer possess dimensions that correspond to the internal dimensions of the area the drug layer is to occupy in the dosage form. Next, the drug layer is placed in contact with the push-displacement layer. The layering of the drug layer and the push-displacement layer can be fabricated by conventional press-layering techniques. The bilayers possess dimensions corresponding to the dimensions of the internal compartment or internal space of the dosage form. Finally, the two-layer compartment forming members are surrounded and coated with an inner and an outer membrane. A passageway is laser drilled or mechanically drilled through the membranes to contact the drug layer, with the dosage form optically oriented automatically by the laser equipment for forming the passageway on the preselected drug surface.

In another manufacture, the dosage form is manufactured by the wet granulation technique. In the wet granulation technique the drug and the ingredients comprising the drug formulation are blended using a solvent, such as isopropyl alcohol, as the granulation fluid. Other granulating fluid, such as water, or denatured alcohol 100% can be used for this purpose. The ingredients forming the drug formulation are individually passed through a 40 mesh screen and then thoroughly blended in a mixer. Next, other ingredients comprising the drug formulation are dissolved in a portion of the granulation fluid, such as the solvent described above. Then, the latter prepared wet blend is slowly added to the drug blend with continual mixing in the blender. The granulating fluid is added until a wet blend mass is produced, which wet mass is then forced through a 20 mesh screen onto over trays. The blend is dried for 18 to 24 hours at 25° C. to 40° C. The dry granules are then screened with for example, a 16 mesh screen. Next, a lubricant is passed through a 60 mesh screen and added to the dry screened granule blend. The granulation is put into milling jars and mixed on a jar mill for 1 to 10 minutes. The first and second layered compositions are pressed into a layered tablet, for example, in a Manesty® layer press.

Another manufacturing process that can be used for providing the drug and push-displacement compositions comprise blending their powdered ingredients in a fluid bed granulator. After the powdered ingredients are dry blended in the granulator, a granulating fluid, for example, poly(vinylpyrrolidone) in a solvent, such as water, is sprayed onto the respective powders. The coated powders are then dried in a granulator. This process coats the ingredients present therein while spraying the granulating fluid. After the granules are dried, a lubricant, such as stearic acid or magnesium stearate, is blended as above into the mixture. The granules are then pressed in the manner described above. In another embodiment, when the fluid granulating process is used to manufacture the push-displacement composition, an antioxident optionally present in the polyalkylene oxide can be removed during the processing step. If antioxidant is desired, it can be added to the push-displacement composition, and this can be accomplished during the fluid bed granulation described above.

The dosage form of this invention is manufactured in another embodiment by mixing a drug with composition-forming ingredients and pressing the composition into a solid layer possessing dimensions that correspond to the internal dimensions of the compartment space adjacent to a passageway. In another embodiment, the drug and other drug composition forming ingredients and a solvent are mixed into a solid, or semi-solid, by conventional methods such as ball-milling, calendaring, stirring, or roll-milling, and then pressed into a preselected, layer-formed shape.

In the general manufactures as presented herein, the manufacture comprising a drug and compositional forming ingredients are placed in contact with the push-displacement layer, and the drug layer and the push layers are surrounded then with the bilayered coats. The layering of the drug composition and the push-displacement composition can be accomplished by using a conventional two-layer tablet press technique. The membranes can be applied by molding, spraying or dipping the pressed shapes into membrane-forming materials. Another technique that can be used for applying the membranes is that air-suspension coating procedure. This procedure consists in suspending and tumbling the two layers in a current of air until the membrane-forming composition are applied separately to the compartment layers. Manufacturing procedures are described in Modern Plastics Encyclopedia, Vol. 46, pp. 62-70 (1969); and in Pharmaceutical Sciences, by Remington, 14th ed., pp. 1626-1948 (1970) published by Mack Publishing Co., Easton, Pa. The dosage form can be manufactured by following the teaching the U.S. Pat. Nos. 4,327,725; 4,612,008; 4,783,337; 4,863,456; and 4,902,514.

### DETAILED DISCLOSURE OF EXAMPLES

The following examples are merely illustrative of the present invention and they should not be considered as limiting the scope of the invention in any way, as these examples and other equivalents thereof will become apparent to those of ordinary skill in the art in the light of the present disclosure and the accompanying claims.

### Example 1

A therapeutic dosage form provided by the invention as prepared as follows: first, 40.0 g of oxybutynin hydrochloride, 180 g of mannitol, and 770 g of polyethylene oxide of 100,000 weight average molecular weight are dry blended for 10 minutes in twin-shell blender. Next, the dry blend drug formulation is blended with 10 g of magnesium stearate, and the blended ingredients thoroughly blended to produce a homogenous formulation. Next, the dry blend drug formulation is compressed into a single layer tablet, comprising 400 mg of oxybutynin formulation, under a pressure of two tons. The compression produced a 7/16 inch (11.1 mm) diameter standard round tablet to provide the oxybutynin formulation, comprising oxybutynin and the pharmaceutically acceptable carrier polyethylene oxide.

Next, the oxybutynin drug formulation is coated with a hydrophilic membrane and a lipophilic membrane. The lipophilic membrane or internal membrane comprises 75 wt % ethyl cellulose and 25 wt % hydroxypropylcellulose flux enhancer in an aqueous dispersion. The lipophilic membrane is applied in a coater to a thickness of 5 mils (0.127 mm). Then, the lipophilic submembrane is overcoated with an exterior hydrophilic membrane, the exterior or outer membrane. The hydrophilic membrane comprises 60 wt % cellulose acetate comprising an acetyl content of 39.8%, micronized to a particle size of 7.5 microns (0.0075 mm), 24 wt % triacetin, 6 wt % Polysorbate® 65 (polyethylene sorbitan fatty and ester consisting of 65 moles of ethylene, and 10 wt % zein of 3.0 microns (0.003 mm). The resulting bimembrane coated sustained release dosage form is dried for 3 days at 50° C. Next an exit passageway is drilled across the bimembranes to provide the therapeutically operative sustained release dosage form.

### Example 2

A sustained release dosage form that delivers a drug at a controlled-rate per zero order time is manufactured by the invention as follows: first 1.2 g of diltiazem hydrochloride, 21.3 g of sorbitol, and 97.4 g of polyethylene oxide of 200,000 weight average molecular weight are dry blended for 7 to 10 minutes in a blender. Next, the dry composition is blended with a 1.8 g of lubricant calcium oleate for 7 minutes to produce a homogenous diltiazem formulation. Then, the diltiazem formulation comprising 180 mg of the diltiazem hydrochloride is compressed under a pressure of two-tons into a round tablet-drug can with a 9/32 inch (7.14 mm) diameter.

Next, the drug-cores are transferred to a coating machine, where they are spray-coated with a membrane forming solution comprising ethylcellulose possessing a 158,000 weight-average molecular weight and hydroxypropylcellulose comprising a 85,000 number-average molecular weight. The coating solvent consist of ethanol and water. The percent ratio of ethylcellulose to hydroxypropylcellulose is 55 to 45 respectively. The coating solution is sprayed around the drug core to apply an inner membrane of 5 mils, (0.127 mm). Next, the inner membrane is overcoated with an outer membrane 2 mil (0.051 mm) thick. The outer membrane comprises cellulose acetate possessing an acetyl content of 38.5% and a 40,000 weight-average molecular weight, triacetin (1,2,3-propanetriol triacetate), micronized gelatin, and polyoxyethylene glycol trirecinoleate (Polyoxyl 35 castor oil Cremophor® EL, commercially available from BASF Corp.) dissolved in acetone. The ratio of cellulose acetate, to triacetin, to micronized gelatin to Polyoxyl 35 castor oil is 65 to 20 to 5 to 10, wt % respectively. The dual membrane coated dosage forms are air dried at 25° C. An exit passageway is drilled through the dual membranes to connect the drug core with the exterior of the dosage form. The dosage form delivers the calcium antagonist at a controlled rate up to 24 hours to a patient to exhibit the influx of calcium ions during biomembrane depolarization of cardiac and vascular smooth muscles for the management of hypertension.

### Example 3

A dosage form for delivering a sustained release dose of drug at a controlled rate continuously up to 24 hours is provided as follows: 500 g of procainamide hydrochloride, a pharmaceutically acceptable carrier comprising 233.8 g of polyethylene oxide of 200,000 molecular weight and 233.8 g of polyethylene oxide of 300,000 molecular weight, and 30 g of hydroxypropylmethylcellulose having a methoxyl content of 29 wt %, a hydroxylpropyl content of 10 wt % and a 11,300 molecular weight, are passed through a mesh screen having 40 wires per inch. The dried powders are tumble mixed for 5 minutes. To the dry mix is added slowly with stirring in an annhydrous ethyl alcohol to form a damp mass. The damp mass is passed through a 20 mesh screen to produce granules that are dried overnight at ambient conditions. After drying, the granules are passed again through a 20 mesh sieve. Then, 2.5 grams of the lubricant magnesium stearate, previously passed through a 60 mesh sieve are tumble mixed for two minutes in a V-blender. This process produces the dry formulation.

An expandable composition, that functions as an osmotic engine that possesses the ability to change from a rested phase to an expanded phase, is prepared as follows: first, 687.5 g of polyethylene oxide of 7,000,000 molecular weight, 50 g of hydroxypropylmethylcellulose, 50 g of polyacrylic and 10 g of ferric oxide are passed through a 40 mesh screen and dry mixed for five minutes. The resulting dry mix is wetted with anhydrous ethyl alcohol and formed into granules. Next, 2.5 g of magnesium stearate is sized through a 60 mesh screen and added to the granules, and mixed for 7 minutes. This manufacture produces the expandable composition.

Next, tablet cores of the dosage form are made by feeding each of the drug formulation and the expandable composition separately to a bilayer tablet press fitted with a standard biconcave 11/32 inch (8.73 mm) round tablet tooling die. The granulations are fed into the machine from individual hoppers. The drug formulation is fed first and it was lightly pretamped to form a lightly compressed mass of 165 mg. Next, the expandable composition, 80 mg, is compressed onto the drug formulation, with a final compression force of about 2 tons, thereby forming a bilayered core. Each core comprises a dose of 75 mg of procainamide hydrochloride.

Next, a batch of the bilayer cores, or matrixes are transferred to a coating machine and sprayed with an interior membrane, followed by an exterior membrane. The interior lipophilic attracting membrane is formed from a solution consisting of 64 g of ethyl cellulose possessing a molecular weight of 220,000 grams per mole with an ethoxyl content of 48.0-49.5 weight percent, and 21 g of hydroxypropylcellulose possessing a molecular weight of 60,000 grams per mole in a solvent comprising 2,400 g of anhydrous ethanol and 120 g of distilled water. The membrane forming solution is sprayed in a current of warm, dry air, until a dry coat weighing 32 mg is deposited onto each bilayer matrix, to provide the first membrane or the interior membrane of the dosage form.

Next, the first interior membrane bilayer drug cores are overcoated with a second, or exterior hydrophilic membrane. The hydrophilic exterior membrane is prepared by blending 8 g of 65 polyoxyethylene sorbitan tristearate consisting of 65 moles of ethylene, 10 g of micronized protein collagen, and 70 g of triacetin dispersed in 2500 ml of distilled water with heat and stirring to provide a homogenous composition. Next, 64 g of cellulose acetate having an acetyl content of 39.8% and a molecular weight of 40,000 g per mole possessing a particle size of 3-5 microns is dispersed into the composition with stirring continuously. Then, a batch of bilayer cores are placed in a fluidized coater and the membrane applied until the second membrane is 2 mils (0.05 mm) thick. This provides the dosage form. Finally, an exit pore is drilled through the two coat to provide a 30 mil (0.75 mm) exit connecting the drug formulation with the exterior of the dosage form.

### Example 4

An extended release dosage form that provide continuous zero order therapy up to 24 hours is provided by following the above examples. The dosage form provided by this example comprises a drug formulation comprising 180 mg of pseudoephedrine, 23 mg of osmagent sodium chloride, 7.4 mg of hydroxypropylemethylcellulose, 24.7 mg of microcrystalline cellulose, 9.9 mg of polyvinylpyrrolidone, and 0.6 mg of magnesium stearate is enveloped with a 5 mil (0.127 mm) lipophilic composition. The composition comprises 50 mg of ethyl cellulose having an ethoxyl content of 48.0-49.5 wt % (weight percent) and a viscosity value of 100 cps. The lipophilic composition comprises 40 mg of the fluid flux enhancer hydroxyproplcellulose. Four of the dosage forms coated with the single membrane were fed to dogs. One dosage form was not recovered as it disintegrated into small pieces. The other three single coated dosage forms were recovered as fragments.

Next, six of the single, lipophilic membrane coated dosage forms were overcoated with a hydrophilic membrane. The over membrane was 2 mils (0.05 mm) thick and the over membrane consists of 40% cellulose acetate having an acetyl content of 39.8% and a viscosity value of 10 seconds. The cellulose acetate was blended with 60% flux enhancers represented by 10% sorbitol, 10% zein and 40% triacetin. In a clinical study, the dual membrane dosage forms next were fed to dogs. The clinical study showed all of the six dual membrane coated dosage forms functioned in vivo and were recovered intact. The clinical study unexpectedly demonstrated that a very thin hydrophilic exterior membrane, such as 2 mils (0.05 mm) of cellulose acetate can protect and prevent a lipophilic submembrane from 1 mil to 20 mil (0.25 mm-0.50 mm) disintegrating in vivo. Also, a thin cellulose acetate membrane unsupported by another and different membrane would collapse in such a clinical test without the mechanical support provided by the lipophilic ethylcellulose inner membrane.

### Example 5

The procedure of the above examples were followed in this example. In this example, the above and same submembrane, the inner membrane, is overcoated with a 2 mil (0.05 mm) over membrane. The present over membrane comprises 60% cellulose acetate micronized to a nominal particle size of 5 to 10 (0.005 to 0.010 mm) microns and sprayed as a composition that comprises 24% triacetin, 10% zein, and 6% polyoxyethylene 20 sorbitan tristearate. The resulting hydrophilic membrane possesses a low resistance to abrasion. In a clinical study, as these dosage forms transit the gastrointestinal tract, the outer interior, membrane shows a low resistance to the peristalic waves of the gastrointestinal tract. The outer membrane's low resistance to in vivo movements results in the outer membrane being abraded away. This process exposes the lipophilic submembrane to disintegration for easy discharge from the gastrointestinal environment of use.

### Example 6

An extended release dosage form was manufactured according to the mode and the manner of the invention. The dosage form of this example comprises a lipophilic inner membrane 5 mils (0.127 mm) thick consisting of 75 wt % ethylcellulose and 25 wt % hydroxypropylcellulose flux enhancer, coated around a drug core comprising a drug layer and an expandable hydrogel layer. The ethylcellulose is a commercially available aqueous dispersion, Surelease® manufactured by Colorcon, Inc., West Point, Pa. The flux enhancer was hydroxypropylcellulose possessing a 80,000 molecular weight. The lipophilic membrane was overcoated with a membrane 2 mils (0.05 mm) thick consisting of 70 wt % cellulose acetate of 39.8% acetyl content micronized to a nominal particle size of 5 to 10 microns, 14 wt % triacetin, 10 wt % micronized albumin, and 6 wt % polyoxyethylene 20 sorbitan tristearate. The dosage form comprising the dual coats was dried for 3 days at 50° C. and an exit port drilled through the dual membranes on the drug layer side.

The dosage forms, were placed next in an aqueous environment. The dosage forms in operation in the aqueous environment hydrodynamically and osmotically delivered the drug layer at a controlled rate with the expandable hydrogel layer remaining in the dosage form. The expandable hydrogel layer exhibited constant internal expanding and swelling that causes the exterior membrane to stretch. The cellulose acetate exterior membrane has a low elongation, it breaks and forms microncracks throughout the membrane as it is stretched in response to applied internal pressure. Next, the lipids, from the environment of use, diffuse into the dosage form and internally soften the interior membrane comprising the ethylcellulose and the hydroxypropylcellulose. The lipophilic membrane, subjected to the peristaltic forces in the gastrointestinal tract, disintegrated accompanied by the development of microcracks in the exterior membrane.

### Example 7

The above examples were followed in this example. In this example, drug cores comprising 25 mg of antiarrhthymic disopyramide phosphate and polyoxyethylene oxide, 100,000 molecular weight, were coated with a 3 mils (0.076 mm) membrane comprising 60 wt % of ethylcellulose, 30 wt % polyoxyethylene 20 sorbitan trioleate and 10 wt % triacetin. In a veterinary clinical study, four of the dosage forms were fed to dogs. Three of the four dosage forms were not recovered, presumably they disintegrated in vivo, and the fourth dosage form was recovered as fragments. Next, dosage forms comprising the identical drug cores, and the identical lipophilic membrane were overcoated with a 2 mil (0.050 mm) hydrophilic membrane comprising 40 wt % cellulose acetate, and 60 wt % flux enhancer, consisting of 30% triacetin, 15% polyethylene glycol of 400 molecular weight %, and 15% micronized globulin having a size of 15 microns or less. In a clinical study, six of these, over-membrane, sub-membrane dosage forms were fed to dogs. All six of the dosage forms were recovered, with three of the dosage forms exhibiting cracks in the overcoat, due to rupture in the membrane resulting from internal swelling pressure generated by the hydrogel in the drug core. The cracks allowed the dosage form, free of its delivered drug, to collapse to a smaller dosage form.

### Example 8

The procedure of example 7 was followed in this example, with all conditions as previously described, except that in this example the outer membrane comprises a blend of cellulose acetate, methylcellulose, and micronized zein.

### Example 9

A dosage form, designed as a sustained release osmotic dosage form, was manufactured comprising an osmotic core comprising a drug layer and an expandable-push contacting layer coated with two different membranes. The dosage forms, after the drug was delivered, underwent a reduction in its mechanical integrity.

The osmotic cores were made by first sieving through a mesh having 40 wires per inch, 500 grams of the antiarrthymic drug, encainide hydrochloride, 233.8 grams of polyethylene oxide having a molecular weight of 200,000 grams per mole, 223.8 grams of polyethylene oxide having a molecular weight of 300,000 grams per mole, and 30 grams of hydroxypropyl methyl cellulose having a molecular weight of 11,300 grams per mole. The polyethylene oxide hydrophilic polymers are available commercially from Union Carbide Inc., Danbury, Conn. The hydroxypropylmethylcellulose polymer possesses an average methoxyl content of 29 weight percent, wt %, and an average hydroxyl content of 10 wt %. The polymer is available commercially from Dow Chemical Co., Midland, Mich. The sieved powders then are tumble mixed in a V-blender thoroughly for 10 minutes and transferred to a planetary mixer. Denatured ethyl alcohol anhydrous, was then added to the powders with stirring to form a uniform damp mass. The damp mass was passed through a sieve having 20 wires per inch, producing elongated extrusions which were air dried at ambient conditions overnight. The dried extrusions were passed through the 20 mesh sieve again, forming free-flowing granules. The dried granules were tumble mixed for 2 minutes with 2.5 grams of magnesium stearate which had previously been passed through a sieve with 60 wires per inch. This processes completed the drug layer granulation.

A second and distinct granulation was prepared by passing through a sieve with 40 wires per inch 687.5 grams of polyethylene oxide having a molecular weight of 7 million, 200 grams of sodium chloride, 50 grams of crosslinked polyacrylic acid, 50 grams of hydroxypropylcellulose equivalent to the hydroxypropylcellulose present in the above granulation, and 10 grams of ferric oxide. The polyethylene oxide is commercially available from Union Carbide Co., Danbury, Conn., and the polyacrylic acid is commercially available from BF Goodrich Co., Cleveland, Ohio. The sized powders were transferred to a planetary mixer and granulated according to the procedures in the previous granulation. The dried granules were tumble mixed with 2.5 grams of 60 mesh magnesium stearate. This completed the push layer granulation. The two granulations were fed to a rotary tablet press fitted with a 3/8 inch (9.5 mm) standard concave round tablet tooling and separate hoppers for each granulation. Bilayer core tablets were compressed with 165 mg of the drug layer granulation and 80 mg of the push layer granulation per tablet. This completed fabrication of the osmotic core. Each drug layer contained a unit dose of 82.5 mg of the drug.

The bilayer cores were transferred to a pharmaceutical pan coater where a membrane was applied to the cores. The coating solution was prepared by dissolving 60 grams of ethyl cellulose, 22 grams of hydroxypropyl cellulose, and 18 grams of polyethylene glycol in a mixture of 2280 grams of ethyl alcohol anhydrous and 120 grams of distilled water. The ethylcellulose had an ethoxyl content of 48.0 to 49.5 weight percent and a molecular weight of 220,000 grams per mole commercially available from Dow Chemical as Ethocel® Standard 100 cps. The hydroxypropyl cellulose had a molecular weight of 80,000 grams per mole available as Klucel E F from Aqualon, Wilmington, Del. The polyethylene glycol was obtained from Union Carbide as Carboxwax 3350. This solution was sprayed onto the cores in a current of warm air to evaporate the solvents until a membrane of three mils (76 microns) thickness was deposited on each core. The resulting batch was dried in forced air at 400 centigrade to remove residual coating solvents.

Drug delivery exits were drilled through the membrane of four of systems using a drill bit. The port had a diameter of 25 mils (0.635 mm) and was positioned at the center of the drug layer. These systems were fed to four dogs which were monitored for 28 hours. The membranes broke up during transit through the animals as evidenced by fragments of the membranes that were recovered in the stools. The release of drug was therefore unpredictable and uncontrolled because timing of the disintegration was not controlled.

Next, the predosage form systems with the ethylcellulose, hydroxypropylcellulose, propylene glycol submembrane were overcoated with two separate membranes. The first membrane was applied in a pharmaceutical pan coater with a membrane dispersion consisting of 64 grams of cellulose acetate having an acetyl content of 39.8%, 86.4 grams of triacetin, 9.6 grams of polysorbate 65 polyethylene 20 sorbitan tristearate, and 17.5 grams of micronzied zein dispersed in 3040 grams of water. The dispersion was prepared by warming the water to 40° centigrade then adding the polysorbate with stirring until it dissolved. The triacetin was then added and dissolved with stirring. The cellulose acetate powder was dispersed concomitantly with the zein into the aqueous mixture, which was allowed to cool to room temperature prior to membrane application. The cellulose acetate had an acetyl content of 39.8 weight percent, and a molecular weight of 40,000 grams per mole and had been milled to a particle size of less than 38 microns (through a mesh having 400 wires per inch). The polysorbate was available from ICI Americas, Wilmington, Del., under the trade name Tween® 65. This aqueous dispersion was applied in a current of warm air until a thickness of 2 mils (51 microns) was applied. Then, a final membrane was applied in the same process. The final membrane was prepared by dissolving 37.8 grams of hydroxypropylmethylcelluose and 4.2 grams of polyethylene glycol 3350 in 378 grams of distilled water. The hydroxypropylmethylcelulose had a methoxyl content of 29 weight percent and a hydroxypropyl content of 10 percent and a molecular weight of 11,900 supplied commercial as Pharmacoat 606®, Shin-Etsu, Tokyo, Japan. This solution was sprayed onto the cores in the pan until a coating thickness of 2 mils (0.0508 mm) was applied. The coated systems were then dried in forced air for 3 days at 500 centigrade. Finally, a delivery exit was drilled into the dosage.

The resulting dosage forms where administered to dogs. In contrast to the four dosage forms with a single membrane consisting of mainly ethylcellulose, which broke-up in the gastrointestinal tracts of the dogs, the four dosage forms with laminiated membranes were recovered intact in the stools of the dogs. The gastrointestinal transit time of the dosage forms were recorded and the amount of residual drug was measured by high pressure liquid chromatography to ascertain the completeness of the dose of drug delivered over time. The transit time and the cumulative percent released is presented in Table 1. The clinical animal study showed the dosage forms with the longest transit times, 49.3 to 50.5 hours delivered 91.9% of dose of drug, and the dosage forms with the shortest transit times of 28.0 to 28.8 hours delivered the lowest dose of drug, 73.5% of the dose. The dosage forms with intermediate transit times delivered more than 73.5% and less than 91.9% of their drug; and three of four of these coats were recovered with cracked membrane. Membranes in the cracked state are more prone to collapse under pressure from the gastrointestinal tract because the viscous hydrogels with the dosage form can ooze more readily from a large crack than from a small exit. The clinical study showed the dosage forms of this invention comprising a laminated bimembrane unexpectedly delivered the prescribed dose of drug in a sustained and controlled rate, and then became collapsible for removal from the gastrointestinal tract. In Table 1, Dosage Form indicates the number of dosage forms, identified as EC CA. The dosage forms comprised dual membranes, an inner membrane identified as EC, indicating the membrane comprising the ethylcellulose, and the outer membrane identified as CA indicating the membrane comprising cellulose acetate. The numbers 1 to 4 indicate the number of dosage forms administered to the animals. Each dosage form comprised 82.5 mg of drug. In the table, % dose delivered 1 to 4 indicate the number of dosage form administered to the animal, each dosage form comprised 82.5 mg of drug, % dose delivered indicates the % delivered by each dosage form, and residual drug indicates the amount of drug left in the dosage form after complete gastrointestinal transit. The clinical results of feeding studies are presented in Table 1.

TABLE 1

| | | Appearance | | |
|---|---|---|---|---|
| Dosage | % Dose | of Coates After | In Vivo | Residual |
| Form | Delivered | In Vivo Transit | Transit Time/Hrs | Drug mg |
| ECCA 1 | 91.9 | Intact | 49.3-50.5 | 6.68 |
| ECCA 2 | 89.5 | Cracked | 30.3-46.0 | 8.69 |
| ECCA 3 | 86.2 | Cracked | 30.3-46.0 | 11.38 |
| ECCA 4 | 73.5 | Cracked | 28.0-28.8 | 21.89 |

### Example 9

A plurality of dosage forms were manufactured by following the procedure of Example 8. First, bilayer drug cores, comprising a drug layer and a expandable-push layer, were transferred to a pharmaceutical pan coater where a membrane was applied to the cores. The coating solution was prepared by dissolving 64 grams of ethyl cellulose, 18 grams of hydroxypropylcellulose, and 18 grams of polyethylene glycol in a mixture of 2280 grams of ethyl alcohol anhydrous, and 120 grams of distilled water. The ethylcellulose had an ethoxyl content of 48.0 to 49.5 weight percent and a molecular weight of approximately 220,000 grams per mole commercially available from Dow Chemical as Ethocel Standard 100 cps. The hydroxypropyl cellulose had a molecular weight of 80,000 grams per mole available as Klucel EF from Aqualon, Wilmington, Del. The polyethylene glycol was obtained from Union Carbide as Carboxwax 3350. This solution was sprayed onto the cores in a current of warm air to evaporate the solvents until a membrane weight of thirty-seven milligrams was deposited on each core. The resulting batch was dried in forced air at 400 centigrade to remove residual coating solvents.

The just applied membrane was overcoated with two separate and distinct but contacting membranes. The first membrane was applied in a pharmaceutical pan coater with a membrane forming dispersion consisting of 24 grams of zein of less than forty-five micron size, 48 grams of micronized cellulose acetate having an acetyl content of 39.8%, 70.4 grams of triacetin, 8.0 grams of polyethylene glycol having a molecular weight of 400 grams per mole, and 9.6 grams of polysorbate 65 dispersed in 3040 grams of water. The dispersion was prepared by warming the water to 45° centigrade then adding the polysorbate with stirring until dissolved. The triacetin was then added and dissolved with stirring. The cellulose acetate powder and zein were then dispersed into the aqueous mixture which was allowed to cool to room temperature prior to coating. The cellulose acetate had the same attributes as described in Example 1. The zein corn protein had a molecular weight of approximately 35,000 grams per mole supplied as grade F5000 from Freeman Industries, Tuckhoe, N.Y. Prior to use it had been air jet mill to a particle size of less than 45 microns. This aqueous coating dispersion was applied to the cores in a current of warm air until a weight of 47.5 mg was applied. Then, a final membrane was applied in the same process. The final membrane was prepared by dissolving 29.4 grams of hydroypropylmethylcellulose and 12.6 grams of polyethylene glycol 8000 in 378 grams of distilled water. The hydroxypropylmethylcellulose had the same attributes as described in Example 8. This solution was sprayed onto the cores in the pan until a membrane weight of 4.2 mg was applied. The membrane-coated dosage forms were dried in forced are for 3 dogs at 50° C. Finally, a delivery exit was drilled into the dosage form to deliver the drug from the dosage form.

Next, three of the just prepared dosage forms were tested in simulated gastric fluid thermostated at 37° centigrade. The fluid was prepared according to the formula specified in the United States Pharmacopeoa 23/National Formulary, 18 page 2053 but without the enzyme, pepsin. The resulting test generated dosage forms which released the drug at controlled rate for extended time. All the dosage forms remained intact throughout the duration of the test. Then, three more dosage forms from the same batch were tested in the same media but with 0.32% pepsin added to the media as well as the lipid, dibutyl sebacate. The three dosage forms again released at controlled rate for extended time. In this test with the added enzyme and lipid, two coats of the three dosage forms developed cracks after twelve hours. This cracking is attributed to the enzymatic attack of the pepsin on the zein which then opened the membrane to lipid sorption by the ethylcellulose, weakening the membrane structure to the point it cracked under the tension produced by the swollen hydrogels from within the core. Thus, in the presence of enzymes normally present in the gastrointestinal tract the rate controlling membrane cracked in situ. Membranes in the cracked state are more prone to collapse under pressure applied from the gastrointestinal tract, because the viscous hydrogels within the core can more readily show form a large open crack than from a small, drilled orifice.

Modifications of the above-described modes for carrying-out the invention are evident to those skilled in the drug-dispensing art in the light of this specification, and they are intended to be within the scope of this invention.

1. A composition indicated for the manufacture of a drug delivery system possessing an extended drug delivery period up to thirty hours, wherein the composition comprises: 35 wt % to 70 wt % of a semipermeable polymer; 10 wt % to 40 wt % of a plasticizer; 20 wt % to 35 wt % of a peptide; and 0 wt % to 10 wt % of a surfactant.

2. A composition indicated for the manufacture of a drug delivery system possessing a sustained-release drug delivery time up to thirty hours, wherein the composition comprises: 35 wt % to 70 wt % of a member selected from the gorup consisting of a cellulose acylate, cellulose diacylate, and a cellulose triacylate polymer; 10 wt % to 40 wt % of a plasticizer that increases the aqueous diffusion coefficient of the composition and is selected from the group consisting of glycerin, triacetin, adipic acid, azelaic acid, citric acid, triethyl citrate, acetyl triethyl citrate, tributyl citrate, acetyl tributyl citrate, butyryl trihexyl citrate, polyethylene glycol, diethylene glycol dipelargonate and triethylene glycol di(2-ethylbutrate); 20 wt % to 35 wt % of a peptide; and 0 wt % to 10 wt % of a surfactant.

3. The composition indicated for the manufacture of drug delivery system according to claim 2, wherein the plasticizer is replaced by a member selected from the group consisting of di-n-butylsebarate, disobutyl phthalate, undecyldocel phthalate and disobutyl phthalate.

4. The composition indicated for the manufacture of a drug delivery system according to claim 2, herein the plasticizer is replaced by a member selected from the group consisting of tricresyl phosphate, cellulose nitrate, dimethylamide, methyl ricinoleate, acetyl triethyl hexyl citrate, methyl phthalyl ethyl glycolate, ethylene glycol diproprionate, monoacetin, diacetin tribulyrin, polyester of diethylene glycol and succinic acid, sorbitol, and diphenylactyl phosphate.

5. A composition for providing a sustained-release dosage form comprising: 35 wt % to 70 wt % of a semipermeable polymer; 10 wt % to 40 wt % of a plasticizer; 20 wt % to 35 wt % of a peptide selected from the group consisting of a protein possessing a molecular weight of 1500 to 350,000; and 0 wt % to 10 wt % of a surfactant selected from the group consisting of an anionic, amphoteric, cationic and nonionic surfactant.

6. The composition for providing a sustained-release dosage form according to claim 5, wherein the peptide comprises a member selected from the group consisting of reticulin, silk, keratin, casein, lactoglobulin, prolamine, gluten, albumin, elastin, soy protein, globulin, gelatin, collagen, and zein.

7. The composition for providing a sustained-release dosage form according to claim 5, wherein the peptide comprises a micron size of 0.01 to 50 microns.

8. A composition for providing an extended-release dosage form comprising: 35 wt % to 70 wt % of a polymer permeable to the passage of an aqueous fluid; 10 wt % to 40 wt % of a plasticizer, 20 wt % to 35 wt % of a compound possessing a peptide band; and 0.01 wt % to 10 wt % of a surfactant selected from the group consisting of an anionic, amphoteric, cationic, and nonionic surfactant, with the weight of ingredients comprising the composition equal to 100 wt %, and the provided extended-release dosage form, when in operation, delivers a drug up to thirty hours.

9. The composition for providing an extended-release dosage form according to claim 8 wherein the surfactant is a member selected from the group consisting of polyoxyethylene sorbitan fatty and, polyoxyethylene sorbitan monolaurate, polyoxyethylene sorbitan monopalmitate, polyoxyethylene sorbitan monostearate, polyoxethylene 20 sorbitan tristearate, polyoxyethylene sorbitan monoleate, polyoxyethylene sorbitan monoisostearate, and polyoxyethlenated stearic acid.

10. A composition for manufacturing a sustained-release dosage form comprising 35 wt % to 70 wt % of a lipophilic-attracting polymer; 25 wt % to 65 wt % of a flux enhancer; and 0 wt % to 10 wt % of a surfactant, with the weight of all materials comprising the composition equal to 100 wt %.

11. A composition for the manufacture of a sustained-release dosage form comprising: 35 wt % to 70 wt % lipophilic-attracting poly(ethyl cellulose) polymer; 25 wt % to 65 wt % of a flux enhancer hydroxyalkylcellulose wherein the alkyl group comprises 1 to 6 carbon atoms; and 0.1 wt % to 10 wt % of a surfactant.

12. The composition for the manufacture of a sustained-release dosage form according to claim 11, wherein the poly(ethyl cellulose) comprising an ethoxyl content of 44 to 51%.

13. The composition for the manufacture of a sustained-release dosage form according to claim 11, wherein the poly(ethyl cellulose) exhibits a visiosity of 3 to 350 centipoise.

14. The composition for the manufacture of a sustained-release dosage form according to claim 11, wherein the hydroxyalkylcellulose is selected from the group consisting of hydroxyethylcellulose and hydroxypropylcellulose.

15. The composition for the manufacture of a sustained-release dosage form according to claim 11, wherein the surfactant comprises a member selected from the group consisting of polyoxyl 4 stearate, polyoxyl 8 stearate, polyoxyl 20 stearate, polyoxyl 30 stearate, polyoxyl 40 stearate, polyoxyl 50 stearate, polyoxyl 100 stearate, polyoxyl 4 distearate and polyoxyl 150 distearate, and wherein the number refers to the surfactant polymer length in oxyethylene units.

16. The composition for the manufacture of a sustained-release dosage form according to claim 11, wherein the surfactant comprises a member selected from the group consisting of polyoxyethylene alkyl ether, polyoxyl 2 cetyl ether and polyoxyl 23 lauryl ether wherein the whole number denotes the number of oxyethylene units.

17. The composition for the manufacture of a sustained-release dosage form according to claim 11, wherein the surfactant comprises a surfactant selected from the group consisting of polyoxyethylene castor oil, polyoxy 35 castor oil, polyoxyl 40 hydrogenated castor oil, and polyoxyethylene sorbitan fatty acid estera.

18. (canceled)

19. (canceled)

20. (canceled)
